SEQUENCE LISTING (8 sequences)

SEQ ID NO: 1 - DNA, 22 bp, Artificial Sequence
  Synthetic
tgtagttgaggtcaatgaaggg

SEQ ID NO: 2 - DNA, 19 bp, Artificial Sequence
  Synthetic
acatcgctcagacaccatg

SEQ ID NO: 3 - DNA, 21 bp, Artificial Sequence
  Synthetic
gctggcttattctgagatgga

SEQ ID NO: 4 - DNA, 21 bp, Artificial Sequence
  Synthetic
cgcgtctctccacatacattc

SEQ ID NO: 5 - DNA, 20 bp, Artificial Sequence
  Synthetic
gaagctggatacggatgtca

SEQ ID NO: 6 - DNA, 22 bp, Artificial Sequence
  Synthetic
cgcctggttactatcaaaagga

SEQ ID NO: 7 - DNA, 20 bp, Artificial Sequence
  Synthetic
gtttgctgcatcgacatctg

SEQ ID NO: 8 - DNA, 22 bp, Artificial Sequence
  Synthetic
ctctggaatatccctggacaac

(19) United States
(12) Patent Application Publication
(10) Pub. No.: US 2024/0058364 A1
(43) Pub. Date: Feb. 22, 2024

(54) A COMPOSITION FOR USE IN THE TREATMENT OF RECTAL AND/OR INTESTINAL INFLAMMATION

(71) Applicant: CANNAMORE BIOTECHS LTD., Bnei-Brak (IL)

(72) Inventor: Sari Prutchi SAGIV, Kibbutz HaMaapil (IL)

(21) Appl. No.: 18/270,017

(22) PCT Filed: Dec. 30, 2021
(86) PCT No.: PCT/IL2021/051560

§ 371 Date: Jun. 28, 2023

Related U.S. Application Data
(60) Provisional application No. 63/132,527, filed on Dec. 31, 2020.

(51) Int. Cl. A61K 31/00; A61K 31/19; A61K 31/728; A61K 31/733; A61P 1/04

(52) U.S. Cl. CPC A61K 31/658; A61K 31/19; A61K 31/728; A61K 31/733; A61P 1/04

(57) ABSTRACT

The present invention is directed to a composition comprising a cannabinoid and hyaluronic acid and a method for use thereof, such as for the treatment and prevention of inflammatory diseases in a subject.

### CROSS-REFERENCE TO RELATED APPLICATIONS

This application is a PCT International Application claiming the benefit of priority of U.S. Patent Application No. 63/132,527, filed Dec. 31, 2020, which is hereby incorporated by reference in its entirety.

### FIELD OF THE INVENTION

The invention relates generally to the field of compositions comprising one or more cannabinoids and is directed to methods of using the same such as for treating of rectal and/or intestinal inflammation.

### BACKGROUND

Cannabidiol (CBD) is a major non-psychotropic constituent of Cannabis. CBD has anti-convulsive, anti-anxiety, anti-psychotic, anti-nausea and anti-inflammatory and immunomodulatory properties. CBD binds to cannabinoid and non-cannabinoid receptors and its mechanism of action is not fully known.

The chemical nomenclature of CBD differs from that of Tetrahydrocannabinol (THC). While the latter has a pyran ring, which determines its numbering, CBD has no heterocyclic ring and its numbering stems from that of the terpene ring. The chemistry of CBD has been well explored over the last 50 years. In view of the various, potentially therapeutic, effects caused by CBD, it seems plausible that novel synthetic approaches will still be developed in the future to lead to new types of derivatives.

Numerous rectal and/or intestinal inflammation related diseases are well known in the art, including inter alia pouchitis and ulcerative colitis. The exact pathophysiological mechanisms of these diseases or disorders remain unknown. Currently, there is no clinically approved treatment for pouchitis and ulcerative colitis. Therefore, there is an ongoing need to develop novel methods of treating or preventing rectal and/or intestinal inflammation diseases.

The foregoing examples of the related art and limitations related therewith are intended to be illustrative and not exclusive. Other limitations of the related art will become apparent to those of skill in the art upon a reading of the specification and a study of the figures.

### SUMMARY

The following embodiments and aspects thereof are described and illustrated in conjunction with systems, tools and methods which are meant to be exemplary and illustrative, not limiting in scope.

In one aspect of the invention, there is a composition comprising (i) a cannabinoid, and (ii) a hyaluronic acid (HA) component; wherein a weight per weight (w/w) concentration of the cannabinoid within the composition is from 0.1 to 50%; and a w/w concentration of the HA component within the composition is from 1 to 90%.

In one embodiment, the cannabinoid comprises at least one of: cannabidiol-C4 (CBD-C4), cannabidiol (CBD-C5), cannabidiol momomethyl ether, cannabidiolic acid (CBD-A), cannabigerolic acid (CBN-A), cannabigerol (CBG), cannabinol (CBN), cannabinolic acid (CBN-A), cannabichromenic acid (CBC-A), cannabichromene (CBC), cannabidivarin, cannabidiorcol, and cannabidivarinic acid or any combination thereof.

In one embodiment, the HA component comprises at least one of: HA, a derivative of HA, a salt of HA or any combination thereof.

In one embodiment, at least 90% w/w of the total cannabinoid content is CBD-C5.

In one embodiment, the HA has an average molecular weight ranging from 1000 to 106 Da.

In one embodiment, a w/w ratio of (i) the cannabinoid to (ii) the HA component within the composition is from 0.1:20 to 1:1.

In one embodiment, the composition further comprises an additional compound comprising a short chain fatty acid (SCF), a fructan or any combination thereof.

In one embodiment, the SCF comprises propionic acid, butyric acid or both, including any salt thereof.

In one embodiment, the fructan comprises inulin, a derivative thereof or both.

In another aspect, there is a pharmaceutical composition, comprising a therapeutically effective amount of the composition of the invention and optionally a pharmaceutically acceptable carrier.

In one embodiment, the pharmaceutical composition is in a form of a suppository or an enema.

In one embodiment, the pharmaceutical composition is for use in the prevention or treatment of a disorder associated with rectal and/or intestinal inflammation.

In one embodiment, the disorder is selected from the group comprising: pouchitis, inflammatory bowel disease, inflammation of the distal ileum, inflammation of the terminal ileum or any combination thereof.

In another aspect, there is a kit comprising a first composition comprising the cannabinoid and a second composition comprising the HA component and/or the SCF, as disclosed herein.

In another aspect, there is a method for preventing or treating a disorder associated with vaginal, rectal and/or intestinal inflammation in a subject in need thereof, comprising administering to the subject the pharmaceutical composition of the invention.

In one embodiment, administering is by a topical administration, a rectal administration or both.

In one embodiment, the pharmaceutical composition comprises a daily dose of CBD-C5 in an amount ranging from 1 to 2,000 mg.

In one embodiment, the pharmaceutical composition comprises a daily dose of an HA in an amount ranging from 1 mg to 100 g.

In addition to the exemplary aspects and embodiments described above, further aspects and embodiments will become apparent by reference to the figures and by study of the following detailed description.

In addition to the exemplary aspects and embodiments described above, further aspects and embodiments will become apparent by reference to the figures and by study of the following detailed description.

### BRIEF DESCRIPTION OF THE DRAWINGS

FIG. 1: is a bar graph showing THP-1 cells viability upon 24 h incubation with 30 μM Cannabidiol (CBD) and 20 mM Sodium Butyrate (NaB) (Combo 1) or with 30 μM Cannabidiol (CBD) and 20 mM Sodium Propionate (NaP) (Combo 2).

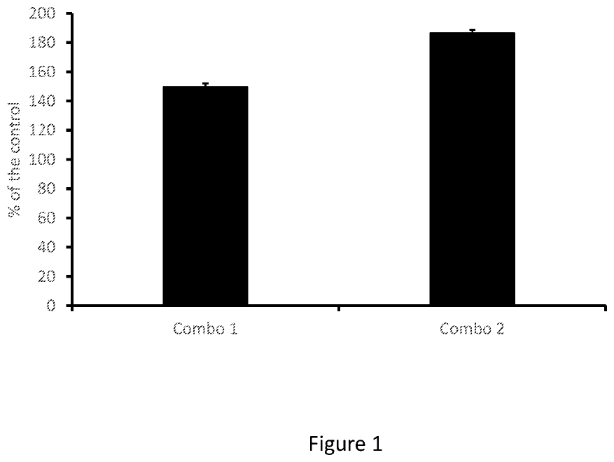

FIG. 2: is a bar graph showing IL8 secretion from LPS-stimulated THP-1 cells upon 24 h incubation with Combo 1 or with Combo 2. The results are shown as ratio between the cells' viability values and the IL8 secretion values, for a same given well.

FIG. 3: is a bar graph showing IL8 secretion from TNFα induced Caco-2 cells upon 24 h incubation with Combo 1 or with Combo 2. The results are shown as percentage of the control.

FIG. 4: is a bar graph showing IL8 secretion from TNFα induced Caco-2 cells upon 24 h incubation with Combo 1 or with Combo 2. The results are shown as ratio between the cells' viability values and the IL8 secretion values, for the same given well.

FIG. 5: is a bar graph showing in-vivo treatment efficiency of the exemplary compositions of the invention (HA/CBD/butyrate, HA/CBD/propionate) compared to controls (saline as a negative control, HA, HA/CBD, HA/butyrate and HA/propionate), as evaluated by histopathology on a dextran sodium sulphate (DSS) induced colitis mice model. In brief, 42 Female c57blk mice were supplemented with different treatments and scored 11 days after treatment for histopathological findings—mucosal ulceration, necrosis, and inflammation. The results are represented as a percentage of the negative control (saline treated animals).

FIG. 6: is a bar graph showing a disease index activity (DAI) at day 6 after treatment with the exemplary compositions of the invention (HA/CBD/but, HA/CBD/pro) compared to controls (saline as a negative control, HA, HA/CBD, HA/but and HA/pro), as evaluated by DSS model.

FIG. 7: is a bar graph showing a colon length of the DSS induced colitis mice treated with the exemplary compositions of the invention (HA/CBD/but, HA/CBD/pro) compared to controls (saline as a negative control, HA, HA/CBD, HA/but and HA/pro) 11 days after treatment.

### DETAILED DESCRIPTION

In one aspect of the invention disclosed herein, there is a composition comprising (i) a cannabinoid, and (ii) a hyaluronic acid (HA) component; wherein:
a weight per weight (w/w) concentration of the cannabinoid within the composition is from 0.1 to 50%;
and a w/w concentration of the HA component within the composition is from 1 to 90%.

In another aspect of the invention, there is a composition comprising (i) a cannabinoid, (ii) a short chain fatty acid (SCF); wherein:
a weight per weight (w/w) concentration of the cannabinoid within the composition is from 0.1 to 10%, or from 0.1 to 50%;
and a w/w concentration of the SCF within the composition is from 0.1 to 10% or form 0.1 to 99%. In some embodiments, the composition further comprises an HA component.

In another aspect, the composition of the invention comprises (i) a cannabinoid including any salt, any tautomer or any derivative thereof; and (ii) a short chain fatty acid (SCF) including any salt, any tautomer or any derivative thereof (e.g. an alkyl ester, an amide); wherein a w/w ratio between the SCF and the cannabinoid is between 10:1 and 1:5, between 10:1 and 1:1, between 10:1 and 8:1, between 10:1 and 3:1, between 8:1 and 3:1, between 8:1 and 6:1, between 8:1 and 5:1, between 5:1 and 1:1, between 6:1 and 1:1, between 6:1 and 3:1, between 6:1 and 2:1, between 3:1 and 1:1, between 1:1 and 1:2, between 1:2 and 1:5, including any range or value therebetween. In some embodiments, a molar ratio between the SCF and the cannabinoid is between 10.000:1 and 1:1, between 1000:1 and 1:1, between 1000:1 and 500:1, between 500:1 and 100:1, between 100:1 and 50:1, between 50:1 and 30:1, between 30:1 and 3:1, between 30:1 and 25:1, between 25:1 and 20:1, between 20:1 and 15:1, between 15:1 and 10:1, between 25:1 and 10:1, between 10:1 and 5:1, between 5:1 and 1:1, between 6:1 and 1:1, between 6:1 and 3:1, between 6:1 and 2:1, between 3:1 and 1:1, between 1:1 and 1:2, between 1:2 and 1:5, including any range or value therebetween.

### Cannabinoids

In some embodiments, the composition comprises one or more cannabinoid at a w/w concentration from 0.1 to 50%. In some embodiments, a w/w concentration of the one or more cannabinoid within the composition is in a range from 0.1 to 0.5%, from 0.5 to 1%, from 1 to 1.5%, from 1.5 to 2%, from 2 to 2.5%, from 2.5 to 3%, from 3 to 4%, from 4 to 5%, from 5 to 6%, from 6 to 7%, from 7 to 8%, from 8 to 9%, from 9 to 10%, from 10 to 50%, from 10 to 15%, from 15 to 20%, from 20 to 25%, from 25 to 30%, from 30 to 35%, from 35 to 40%, from 40 to 45%, from 45 to 50%, including any range or value therebetween. In some embodiments, a w/w concentration of the one or more cannabinoid within the composition is in a range from 0.001 and 0.1%, from 0.001 and 0.01%, from 0.01 and 0.1%, including any range or value therebetween.

In some embodiments, a w/w concentration of the one or more cannabinoid within the composition is less than 50%, less than 40%, less than 30%, less than 20%, less than 10%, less than 8%, less than 6%, less than 5%, less than 4%, less than 3%, less than 2%, less than 1%, less than 0.5%, less than 0.1%, less than 0.05%, less than 0.01%, including any range or value therebetween.

As used herein, the term “cannabinoid” comprises a cannabidiol (CBD), tetrahydrocannabinol (THC), or any combination of THC and CBD. As used herein, the term “CBD” comprises CBD also termed 2-[(6R)-3-Methyl-6-prop-1-en-2-yl-1cyclohex-2-envyl]-5pentylbenzene-1,3-diol (also referred to as CBD-C5), any chemical derivative (such as constitutional isomers or stereoisomers) thereof or both. As used herein, the term “THC”, comprises THC also termed (−)-trans-Δ⁹-tetrahydrocannabinol, any chemical derivative thereof (such as constitutional isomers or stereoisomers) or both. As used herein the term “chemical derivative” in some embodiments thereof, is related to any chemical isomer such as a structural or a constitutional isomer, a stereoisomer, and a conformer or any combination thereof.

In some embodiments, the composition comprises CBD and is devoid of THC. In some embodiments, one or more cannabinoids consists of CBD. In some embodiments, one or more cannabinoids comprises CBD. In some embodiments, the composition as described herein is devoid of a psychoactive effect.

Non-limiting examples of CBD or chemical derivative thereof include, but are not limited to: cannabidiol-C4 (CBD-C4), CBD-C5, O-acetylated CBD-C5, cannabidiol momomethyl ether, cannabidiolic acid (CBD-A), O-acetylated CBD-A, an ester of CBD-A, cannabigerolic acid (CBN-A), an ester of CBN-A, O-acetylated CBN-A, cannabigerol (CBG), cannabinol (CBN), cannabinolic acid (CBN-A), cannabichromenic acid (CBC-A), cannabichromene (CBC), cannabidivarin, cannabidiorcol, and cannabidivarinic acid including corresponding stereoisomers or any combination thereof.

In some embodiments, the cannabinoid comprises a chemical derivative of CBD, comprising a metabolite of CBD such as but not limited to: (−)-7-hydroxy-CBD and (−)-CBD-7-oic acid and their dimethylheptyl (DMH) homologs, as well as of the corresponding compounds in the enantiomeric (+)-CBD series.

In some embodiments, a chemical derivative of CBD is characterized by a structure wherein at least one of the hydroxyl substituent groups is converted into a functional group such as keto-, alkoxy-, amide-, halo- and carboxy-group. In one embodiment, a CBD derivative is an endocannabinoid derivative. In some embodiments, a CBD derivative is described in Frank D King; G Lawton; A W Oxford Progress in medicinal chemistry. Vol. 44. Pages 207-331, Elsevier Science, 2006 ISBN: 0080462103 9780080462103 which is hereby incorporated by reference in its entirety.

Non-limiting examples of THC or chemical derivative thereof include but are not limited to: (−)-Δ⁹-tetrahydrocannabinol (THC), 2-carboxy-THC (THCA), an ester of THCA, 4-carboxy-THC (THCA-B), an ester of THCA-B, 11-OH-THCA, THC-11-oic acid, Δ8-THC-11-oic acid, 1,1-dimethylheptyl-Δ8-THC-7-oic acid including corresponding stereoisomers or any combination thereof.

In some embodiments, the composition comprises CBD or any chemical derivative thereof and is substantially devoid of THC. In one embodiment, a composition comprising CBD as described herein is substantially devoid of THC. As used herein, CBD or any chemical derivative thereof, according to some embodiments, refers to compounds and/or compositions comprising at least 80% CBD, at least 90% CBD, at least 92% CBD, at least 95% CBD, at least 97%, or at least 99% CBD or any chemical derivative thereof, including any value and range therebetween. Each possibility represents a separate embodiment of the invention. In some embodiments, CBD or any chemical derivative thereof, refers to compounds and/or compositions comprising 80-90% CBD, 85-95% CBD, 92-99% CBD, or 93-100% CBD including any value and range therebetween. Each possibility represents a separate embodiment of the invention.

In some embodiments, substantially devoid of THC refers to the % by weigh. In some embodiments, substantially devoid of THC is less than 10% by weight, less than 7% by weight, less than 5% by weight, less than 3% by weight, less than 1% by weight, less than 0.5% by weight, less than 0.3% by weight, or less than 0.1% by weight THC, or any value and range therebetween. Each possibility represents a separate embodiment of the invention. In some embodiments, substantially devoid of THC is 0.1-0.5% by weight, 0.3-3% by weight, 1-5% by weight, 3-7% by weight, 6-10% by weight THC, or any value and range therebetween. Each possibility represents a separate embodiment of the invention.

In some embodiments, a composition as described herein comprises less than 20% w/w THC. In some embodiments, a composition as described herein comprises less than 15% w/w THC. In some embodiments, a composition as described herein comprises less than 10% w/w THC. In some embodiments, a composition as described herein comprises less than 5% w/w THC. In some embodiments, a composition as described herein comprises less than 3% w/w THC. In some embodiments, a composition as described herein is devoid or substantially devoid of a psychoactive effect.

In some embodiments, the composition comprises CBD-C5.

The chemical structure of CBD-C5 is shown hereinbelow in Formula I:

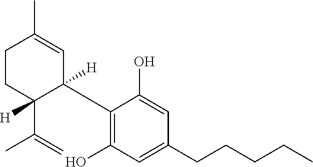 [SMILES: C=C(C)[C@@H]1CCC(C)=C[C@H]1c1c(O)cc(CCCCC)cc1O]

### Short Chain Fatty Acid

In some embodiments, the composition comprises a short chain fatty acid (SCF), a salt of SCF, a chemical derivative of SCF or any combination thereof. As used herein the term SCF relates to aliphatic carboxylic acids, wherein the aliphatic chain comprises from 1 to 10, from 1 to 2, from 2 to 4, from 4 to 5, from 5 to 7, from 7 to 10, or 1, 2, 3, 4, 5, 6 or 7 carbon atoms including any range between.

In some embodiments, a derivative of the SCF comprises a hydroxylated fatty acid (e.g. alpha-hydroxy carboxylic acid, such as lactic acid), a carboxylated fatty acid (e.g. dicarboxylic fatty acid), and/or a halogenated fatty acid.

Non-limiting examples of SCF include, but are not limited to: acetic acid, propionic acid, butyric acid, valeric acid or any combination thereof.

In some embodiments, the SCF is butyric acid, a salt thereof or both. In some embodiments, the SCF is propionic acid, a salt thereof or both.

Without being bound to any particular theory or mechanism, butyric and/or propionic acid are known in the art as having a beneficial effect on inflammatory processes since it potentiates epithelial barrier function, increases mucin synthesis, inhibits adhesion of leukocytes to endothelium and reduces local production of pro-inflammatory mediators such as TNF-α, NO, IFN-γ, IL-2 and IL-12 while increasing that of anti-inflammatory cytokines. Specifically, butyric acid in some embodiments, has been shown to be effective in the treatment of inflammatory diseases of the gastrointestinal tract (such as inflammatory bowel disease (IBD), colorectal functional and motor disturbances, and neoplasia). Thus, butyric acid in some embodiments, may be beneficial for use in a composition directed to the treatment of a disease or a disorder associated with rectal and/or intestinal inflammation.

In some embodiments, a w/w concentration of the SCF within the composition is from 0.1 to 50%, from 0.1 to 1%, from 1 to 5%, from 5 to 10%, from 10 to 15%, from 15 to 20%, from 20 to 30%, from 30 to 40%, from 40 to 50%, from 0.1 to 50%, including any range or value therebetween. Each possibility represents a separate embodiment of the present invention.

### Hyaluronic Acid

In some embodiments, the composition comprises or further comprises a hyaluronic acid (HA) component at a w/w concentration from 1 to 90%.

In some embodiments, a w/w concentration of an HA component within the composition is in a range from 1 to 1.5%, from 1.5 to 2%, from 2 to 2.5%, from 2.5 to 3%, from 3 to 4%, from 4 to 5%, from 5 to 6%, from 6 to 7%, from 7 to 8%, from 8 to 9%, from 9 to 10%, from 10 to 15%, from 15 to 20%, from 20 to 25%, from 25 to 30%, from 30 to 35%, from 35 to 40%, from 40 to 45%, from 45 to 50%, from 45 to 50%, from 50 to 60%, from 60 to 70%, from 70 to 80%, from 80 to 90%, including any range or value therebetween.

In some embodiments, a w/w concentration of the HA component within the composition is less than 90%, is less than 80%, is less than 70%, is less than 60%, is less than 50%, is less than 40%, is less than 30%, is less than 20%, is less than 10%, is less than 8%, is less than 6%, is less than 5%, is less than 4%, is less than 3%, is less than 2%, is less than 1%, including any range or value therebetween.

As used herein, HA component comprises hyaluronic acid (HA), a salt or a derivative of HA, or any combination thereof. As used herein, HA relates to a polysaccharide, comprising repeating units of D-glucuronic acid and N-acetyl-glucosamine bound via a glycosidic bond. As used herein, a salt of HA relates to D-glucuronic acid in the deprotonated form (i.e. as a carboxylate salt).

As used herein, a derivative of HA relates to chemically modified HA. In some embodiments, a chemically modified HA comprises a side chain modification (e.g. acetylation of a hydroxy group, decarboxylation, esterification or amidation of a carboxy group). In some embodiments, a chemically modified HA comprises one or more of the side chain modifications. In some embodiments, the modifications are the same. In some embodiments, the modifications are different. In some embodiments, a chemically modified HA comprises a combination of modified side chains.

In some embodiments, the HA component is characterized by an average molecular weight from 1000 to 15×106 Da, 1000-10,000 Da, 10,000-20,000 Da, 20,000-30,000 Da, 30,000-40,000 Da, 40,000-50,000 Da, 50,000-100,000 Da, 100,000-200,000 Da, 150,000-400,000 Da, 400,000-1,000,000 Da, 400,000-500,000 Da, 500,000-600,000 Da, 600,000-700,000 Da, 700,000-800,000 Da, 800,000-1,000,000 Da, 1,000,000-1,500,000 Da, 1,500,000-2,000,000 Da, 2,000,000-3,000,000 Da, 3,000,000-5,000,000 Da, 5,000,000-7,500,000 Da, 7,500,000-10,000,000 Da, 10,000,000-12,000,000 Da, 12,000,000-15,000,000 Da, 4,000,000-10,000,000 Da, or 7,500,000-15,000,000 Da, including any range or value therebetween. Each possibility represents a separate embodiment of the present invention.

As used herein, the term “ average molecular weight” encompasses any one of the average weight values selected from: Mn (Number average molar mass), NAMW (Number Average Molecular Weight), Mw (Mass average molar mass), WAMW (Weight Average Molecular Weight), Mz (Z average molar mass), My (Viscosity average molar mass), and MWCO (molecular weight cut-off). Unless stated otherwise this term refers to Mw.

As used herein, HA component, according to some embodiments, refers to compounds and/or compositions comprising the same in an amount of at least 80% by weight, at least 90% by weight, at least 92% by weight, at least 95% by weight, at least 97% by weight, or at least 99% by weight of the HA component, including any value and range therebetween. Each possibility represents a separate embodiment of the invention.

In some embodiments, the HA component comprises one or more HA chains. In some embodiments, “one or more” is two. In some embodiments, one or more HA chains are crosslinked. In one embodiment, cross-linking is inter-crosslinking. As defined herein, the term “inter” refers to the formation of a bond between two moieties residing in two different chains, as opposed to the formation of an “intra” bond between two residues residing within the same chain. In some embodiments, crosslinking of one or more HA chains is via a linker.

A “linker” as defined herein refers to a molecule or macromolecule serving to connect different moieties or functional groups of one or more HA chains. In one embodiment, a linker may also facilitate other functions, including, but not limited to, preserving biological activity, maintaining interactions, and others.

### Composition

In some embodiments, the composition of the invention comprises the cannabinoid and the HA component at a w/w ratio from 0.1:20 to 1:1. In some embodiments, a w/w ratio of the cannabinoid to the HA component within the composition is from 1:200 to 1:150, from 1:150 to 1:100, from 1:100 to 1:50, from 1:50 to 1:40, from 1:40 to 1:30, from 1:30 to 1:20, from 1:20 to 1:10, from 1:10 to 1:8, from 1:8 to 1:5, from 1:5 to 1:2, from 1:2 to 1:1, including any range or value therebetween. Each possibility represents a separate embodiment of the present invention.

In some embodiments, a w/w ratio of the cannabinoid to the HA component within the composition is from 100:1 to 1:1, from 100:1 to 50:1, from 50:1 to 30:1, from 30:1 to 20:1, from 20:1 to 10:1, from 10:1 to 8:1, from 8:1 to 5:1, from 5:1 to 3:1, from 3:1 to 2:1, from 2:1 to 1:1, including any range or value therebetween. Each possibility represents a separate embodiment of the present invention.

In some embodiments, the composition further comprises a fructan. Non-limiting examples of fructans include but are not limited to fructose polysaccharides, fructose oligosaccharides, and inulin. In some embodiments, the composition further comprises inulin. In some embodiments, the composition further comprises a chemical derivative of inulin (such as a cross-linked inulin, an acetylated inulin, an alkylated inulin or any combination thereof). In some embodiments, the composition further comprises sodium benzoate.

In some embodiments, a w/w concentration of a fructan within the composition is from 0.1 to 50%, from 0.1 to 1%, from 1 to 5%, from 5 to 10%, from 10 to 15%, from 15 to 20%, from 20 to 30%, from 30 to 40%, from 40 to 50%, from 0.1 to 50%, including any range or value therebetween. Each possibility represents a separate embodiment of the present invention.

In some embodiments, the composition of the invention comprises or consist essentially of the cannabinoid (e.g. cannabidiol) and one or more SCF (e.g. propionic acid and/or butyric acid). In some embodiments, the composition of the invention comprises or consist essentially of the cannabinoid (e.g. cannabidiol), HA, and one or more SCF (e.g. propionic acid and/or butyric acid). In some embodiments, the composition of the invention is substantially devoid of HA. In some embodiments, the composition of the invention comprises or consist essentially of the cannabinoid (e.g. cannabidiol) and one or more SCF (e.g. propionic acid), wherein a w/w ratio between the SCF and the cannabinoid is between 1000:1 and 1:1, between 1000:1 and 100:1, between 100:1 and 50:1, between 50:1 and 30:1, between 30:1 and 20:1, between 20:1 and 10:1, between 10:1 and 5:1, between 5:1 and 3:1, between 3:1 and 1:1, between 1:1 and 1:2, between 1:2 and 1:5, including any range or value therebetween. Each possibility represents a separate embodiment of the present invention.

In some embodiments, the composition of the invention comprises or consist essentially of the cannabinoid (e.g. cannabidiol) and one or more SCF. In some embodiments, the composition of the invention comprises or consist essentially of the cannabinoid (e.g. cannabidiol) and one or more SCF (e.g. propionic acid), wherein the molar ratio between the SCF and the cannabinoid is between 5000:1 and 5:1, between 5000:1 and 1000:1, between 1000:1 and 500:1, between 500:1 and 100:1, between 100:1 and 50:1, between 50:1 and 30:1, between 30:1 and 5:1, between 1000:1 and 500:1, between 1000:1 and 5:1, between 100:1 and 1:100, 50:1 and 1:50, 10:1 and 1:10, 5:1 and 3:1, between 3:1 and 1:1, between 1:1 and 1:2, between 1:2 and 1:5, including any range or value therebetween. Each possibility represents a separate embodiment of the present invention.

In some embodiments, a w/w concentration of the SCF within the composition (e.g. enema formulation) is from 0.1 to 0.5%, from 0.1 to 0.2%, from 0.2 to 0.4%, from 0.4 to 0.5%, including any range or value therebetween. Each possibility represents a separate embodiment of the present invention.

In some embodiments, a w/w concentration of the SCF within the composition is less than 50%, less than 40%, less than 30%, less than 20%, less than 10%, less than 5%, less than 3%, less than 1%, less than 0.5%, including any range or value therebetween. Each possibility represents a separate embodiment of the present invention.

As used herein, any of butyric acid and/or propionic acid or any salt thereof, according to some embodiments, refers to compounds and/or compositions comprising the same in an amount of at least 80% by weight, at least 90% by weight, at least 92% by weight, at least 95% by weight, at least 97% by weight, or at least 99% by weight of butyric acid and/or propionic acid or any salt thereof, including any value and range therebetween. Each possibility represents a separate embodiment of the invention.

In some embodiments, the composition comprises the cannabidiol, the HA component, and the SCF. In some embodiments, the composition comprises CBD-C5, the HA component, and the SCF. In some embodiments, the composition comprises CBD-C5, the HA component, and butyric acid. In some embodiments, the composition comprises (i) CBD-C5 at a w/w concentration from 0.1 to 50%, from 0.1 to 0.5%, from 0.5 to 1%, from 1 to 5%, from 5 to 10%, including any range between (ii) the HA component at a w/w concentration from 1 to 90%, from 1 to 2%, from 2 to 3%, from 3 to 5%, from 5 to 10%, including any range between and (iii) an additional compound comprising at least one of inulin, a chemical derivative of inulin, SCF (butyric acid, a salt of butyric acid, propionic acid and/or a salt thereof) or any combination thereof at a w/w concentration from 0.1 to 50%, from 0.1 to 0.5%, from 0.5 to 1%, from 1 to 5%, from 5 to 10%, including any range between.

In some embodiments, the composition or kit is characterized by a storage stability of at least 1 week, at least 1 month (m), at least 5 m, at least 10 m, at least 12 m, at least 15 m, at least 20 m, at least 24 m, at least 3 years including any range between.

In some embodiments, storage stability of the composition or of the kit is referred to a prolonged storage thereof under suitable storage conditions comprising inter alia: ambient atmosphere and a temperature of less than 60° C., less than 50° C., less than 40° C., less than 30° C., less than 20° C., less than 15° C., less than 10° C., less than 5° C., less than 2° C., less than 0° C., including any range between.

In some embodiments the term “stable”, refers to the ability of the composition or of the kit to maintain substantially its physical intactness, such as being substantially devoid of degradation, and/or phase separation; and/or to maintain substantially its therapeutical function as described herein. In some embodiments, the composition or of the kiis referred to as stable when it maintains at least 70%, at least 80%, at least 85%, at least 90%, at least 95%, at least 97% including any range between, of the initial biological activity under suitable storage conditions and for a time period described herein.

### Pharmaceutical Composition

According to some embodiments, there is provided a pharmaceutical composition comprising as an active ingredient a therapeutically effective amount of (i) the cannabinoid, (ii) the HA component, and a pharmaceutically acceptable carrier. In some embodiments, the composition further comprises sodium benzoate.

According to another embodiments, there is provided a pharmaceutical composition comprising as an active ingredient a therapeutically effective amount of (i) the cannabinoid and/or a salt thereof and/or a derivative thereof, (ii) the SCF (e.g. propionic acid, butyric acid or both) and/or a salt thereof, and/or a derivative thereof, and a pharmaceutically acceptable carrier.

In some embodiments, the compounds (e.g. the active ingredients and/or excipient or carrier) of the pharmaceutical composition or kit of the invention are pharmaceutical grade compounds.

In some embodiments, the pharmaceutical composition comprises a synergistically effective amount of (i) the cannabinoid and/or a derivative thereof, and of (ii) the SCF (e.g. propionic acid, butyric acid or both) and/or a salt thereof, and/or a derivative thereof. In some embodiments, the synergistically effective amount is sufficient for inducing a statistically significant synergistic therapeutic effect upon administering the composition/combination of the invention to the subject, as compared to alone treatment with (i) or (ii). In some embodiments, the pharmaceutical composition (e.g. enema) comprises (i) CBD-C5 and (ii) SCF (butyric acid, a salt of butyric acid, propionic acid and/or a salt thereof, or any combination thereof) at a w/w ratio between (ii) and (i) at a w/w range between 10:1 and 1:5, between 10:1 and 1:1, between 10:1 and 8:1, between 10:1 and 3:1, between 8:1 and 3:1, between 8:1 and 6:1, between 8:1 and 5:1, between 5:1 and 1:1, between 6:1 and 1:1, between 6:1 and 3:1, between 6:1 and 2:1, between 3:1 and 1:1, between 1:1 and 1:2, between 1:2 and 1:5, including any range or value therebetween, including any range between, and optionally (iii) the HA component at a w/w concentration from 1 to 2%, from 2 to 3%, from 3 to 5%, from 5 to 10%, including any range between.

In some embodiments, the pharmaceutical composition (e.g. enema) comprises (i) CBD-C5 at a w/w concentration from 0.1 to 0.5%, from 0.5 to 1%, from 1 to 5%, from 5 to 10%, including any range between (ii) SCF (butyric acid, a salt of butyric acid, propionic acid and/or a salt thereof) or any combination thereof at a w/w concentration from 0.1 to 0.5%, from 0.5 to 1%, from 1 to 5%, from 5 to 10%, including any range between, and optionally (iii) the HA component at a w/w concentration from 1 to 2%, from 2 to 3%, from 3 to 5%, from 5 to 10%, including any range between.

In some embodiments, each dosage form (e.g. enema) comprises between about 60 and about 300 mg of the cannabinoid (e.g. CBD-C5) such as between about 100 and 200 mg, or about 200 mg; and from about 100 to about 900 mg, from about 100 to about 2000 mg, from about 100 to about 500 mg, from about 500 to about 1000 mg, from about 1000 to about 1500 mg, from about 1000 to about 2000 mg, from about 100 to about 800 mg, from about 100 to about 700 mg, from about 200 to about 900 mg, from about 200 to about 700 mg, or about 165 mg of the SCF (e.g. propionic acid and/or butyric acid) and/or any salt thereof. In some embodiments, each dosage form (e.g. enema) further comprises HA (e.g. as an aqueous HA solution) at a concentration of about 2% w/w.

In some embodiments, the pharmaceutical composition comprises as an active ingredient a therapeutically effective amount of a cannabidiol and the HA component with an acceptable carrier. In some embodiments, the pharmaceutical composition comprises as an active ingredient a therapeutically effective amount of: (i) CBD-C5, (ii) the HA component, and (iii) an additional compound comprising butyric acid, inulin or both. In some embodiments, the pharmaceutical composition comprises (i) CBD-C5 at a w/w concentration from 0.1 to 50%, (ii) the HA component at a w/w concentration from 1 to 90%, and (iii) an additional compound comprising any of SCF (e.g. butyric acid and/or propionic acid and/or any salt thereof), inulin or any combination thereof at a w/w concentration from 0.1 to 50%.

In one embodiment, the w/w ratio of the HA component to the additional compound in the composition as described herein is 50:1 to 1:2. In one embodiment, the w/w ratio of the HA component to the additional compound in the composition as described herein is 50:1 to 2:1. In one embodiment, the w/w ratio of the HA component to the additional compound in the composition as described herein is 50:1 to 10:1. In one embodiment, the w/w ratio of the HA component to the additional compound in the composition as described herein is 50:1 to 30:1. In one embodiment, the w/w ratio of the HA component to the additional compound in the composition as described herein is 30:1 to 1:1. In one embodiment, the w/w ratio of the HA component to the additional compound in the composition as described herein is 30:1 to 5:1.

In one embodiment, the w/w ratio of the HA component to the additional compound in the composition as described herein is 50:1 to 1:2. In one embodiment, the w/w ratio of the HA component to butyric acid or a salt thereof in the composition as described herein is 50:1 to 2:1. In one embodiment, the w/w ratio of the HA component to butyric acid or a salt thereof in the composition as described herein is 50:1 to 10:1. In one embodiment, the w/w ratio of the HA component to butyric acid or a salt thereof in the composition as described herein is 50:1 to 30:1. In one embodiment, the w/w ratio of the HA component to butyric acid or a salt thereof in the composition as described herein is 30:1 to 1:1. In one embodiment, the w/w ratio of the HA component to butyric acid or a salt thereof in the composition as described herein is 30:1 to 5:1.

In one embodiment, the w/w ratio of inulin to butyric acid or a salt thereof in the composition as described herein is 50:1 to 1:2. In one embodiment, the w/w ratio of inulin to butyric acid or a salt thereof in the composition as described herein is 50:1 to 2:1. In one embodiment, the w/w ratio of inulin to butyric acid or a salt thereof in the composition as described herein is 50:1 to 10:1. In one embodiment, the w/w ratio of inulin to butyric acid or a salt thereof in the composition as described herein is 50:1 to 30:1. In one embodiment, the w/w ratio of inulin to butyric acid or a salt thereof in the composition as described herein is 30:1 to 1:1. In one embodiment, the w/w ratio of inulin to butyric acid or a salt thereof in the composition as described herein is 30:1 to 5:1.

As used herein, the term “pharmaceutically acceptable” means approved by a regulatory agency or listed in the U.S. Pharmacopeia or other generally recognized pharmacopeia for use in animals, and more particularly in humans.

As used herein, the term “carrier” refers to a diluent, adjuvant, excipient, or a vehicle administered together with the active ingredient. In some embodiments, the carrier improves the stability of the active ingredient in a living organism. In some embodiments, the carrier improves the stability of the active ingredient within the pharmaceutical composition. In some embodiments, the carrier enhances the bioavailability of the active ingredient.

In some embodiments, carriers are sterile liquids such as water-based liquids; oils, including those of petroleum, animal, vegetable or synthetic origin, such as peanut oil, soybean oil, mineral oil, sesame oil and the like; solvents such as polyethylene glycols, glycerin, propylene glycol, ethanol or other synthetic solvents. In some embodiments, the composition further comprises sodium benzoate.

Other non-limiting examples of carriers include, but are not limited to: terpenes derived from Cannabis, or total terpene extract from Cannabis plants, terpenes from coffee or cocoa, mint-extract, eucalyptus-extract, citrus-extract, tobacco-extract, anis-extract, any vegetable oil, peppermint oil, d-limonene, b-myrcene, a-pinene, linalool, anethole, a-bisabolol, camphor, b-caryophyllene and caryophyllene oxide, 1,8-cineole, citral, citronella, delta-3-carene, farnesol, geraniol, indomethacin, isopulegol, linalool, unalyl acetate, b-myrcene, myrcenol, 1-menthol, menthone, menthol and neomenthol, oridonin, a-pinene, diclofenac, nepafenac, bromfenac, phytol, terpineol, terpinen-4-ol, thymol, and thymoquinone.

In some embodiments, suitable pharmaceutical excipients include starch, glucose, lactose, sucrose, gelatin, malt, rice, flour, chalk, silica gel, salts (e.g., sodium chloride, sodium stearate, and glycerol monostearate), talc, dried skim milk, glycerol, propylene glycol, water, ethanol and the like. The composition may further comprise wetting and/or emulsifying agents, or pH buffering agents such as acetates, citrates or phosphates.

In some embodiments, preservatives such as benzyl alcohol, benzoic acid (including any salt thereof, such as sodium benzoate) and/or methyl parabens; antioxidants (such as ascorbic acid, propyl gallate, tocopherols, tertiary butylhydroquinone, butylated hydroxyanisole, sodium pyrosulfite, potassium pyrosulfite, and butylated hydroxytoluene, or sodium bisulfate); and agents for the adjustment of tonicity (such as sodium chloride or dextrose) are also envisioned.

In some embodiments, the carrier comprises, in total, from about 0.1% to about 99.99999% by weight of the pharmaceutical composition presented herein.

In some embodiments, the pharmaceutical composition includes incorporation of any one the active ingredients into or onto particulate preparations of polymeric compounds such as polylactic acid, polyglycolic acid, hydrogels, etc., or onto liposomes, microemulsions, micelles, unilamellar or multilamellar vesicles, erythrocyte ghosts, or spheroplasts. Such compositions may influence the physical state, solubility, stability, rate of in vivo release, and rate of in vivo clearance.

In some embodiments, the pharmaceutical composition is a liquid at a temperature between 15 to 45° C. In some embodiments, the pharmaceutical composition is a solid at a temperature between 15 to 45° C. In some embodiments, the pharmaceutical composition is a semi-liquid at a temperature between 15 to 45° C. It should be understood that the term “semi-liquid”, is intended to mean materials which are flowable under pressure and/or shear force. In some embodiments, semi-liquid compositions include creams, ointments, gel-like materials and other similar materials. In some embodiments, the pharmaceutical composition is a semi-liquid composition, characterized by a viscosity in a range from 31,000-800,000 cps.

In some embodiments, the pharmaceutical composition is in a form of an ointment, a cream, a gel, a paste, a foam, a suppository, an enema, a pessary, a pad or a gelled stick.

In another aspect of the invention, there is a suppository for use in the prevention or treatment of a disorder associated with rectal, vaginal, urethral and/or intestinal inflammation or disease. In some embodiments, the suppository comprises a cannabinoid and an HA component, wherein the cannabinoid and the HA component are as described hereinabove. In some embodiments, the suppository comprises (i) a cannabinoid at a w/w concentration from 0.1 to 50%, and (ii) the HA component at a w/w concentration from 1 to 90%. In some embodiments, the suppository further comprises and butyric acid a salt thereof or both, as described hereinabove.

In some embodiments, the suppository comprises (i) cannabidiol (CBD) at a w/w concentration from 0.1 to 50%, and (ii) the HA component at a w/w concentration from 1 to 90%, wherein CBD is as described hereinabove.

In some embodiments, the suppository or the composition comprises (i) CBD-C5 at a w/w concentration from 0.1 to 50%, and (ii) the HA component at a w/w concentration from 1 to 90%, from 1 to 5%, from 5 to 10%, from 10 to 20%, from 1 to 10%, from 15 to 25%, from 20 to 30%, from 30 to 50%, from 40 to 50%, from 15 to 30%, from 10 to 30%, from 10 to 40%, from 10 to 50%, from 20 to 50%, from 20 to 40%, from 20 to 60%, from 20 to 70%, from 20 to 80%, from 20 to 90%, from 30 to 60%, from 30 to 70%, from 30 to 80%, from 40 to 60%, from 40 to 70%, from 40 to 80%, from 40 to 90%, from 50 to 60%, from 60 to 70%, from 60 to 90%, from 70 to 90% of the HA component, including any range or value therebetween.

In some embodiments, CBD or any derivative or analog thereof constitutes at least 80% w/w of the cannabinoid or cannabinoids content of the composition. In some embodiments, CBD or any derivative or analog thereof constitutes at least 85% w/w of the cannabinoid or cannabinoids content of the composition. In some embodiments, CBD or any derivative or analog thereof constitutes at least 90% w/w of the cannabinoid or cannabinoids content of the composition. In some embodiments, CBD or any derivative or analog thereof constitutes at least 95% w/w of the cannabinoid or cannabinoids content of the composition.

As used herein, the term “suppository” relates to a solid body of various weights and shapes, adapted for introduction into the rectal, vaginal, or urethral orifice of the human body. In one embodiment, a suppository of the invention melts, softens, and/or at least partially dissolves at body temperature (e.g. about 35° C., or about 40° C. including any range between) or once in contact with a tissue of the subject. A suppository, in some embodiments thereof, is a protectant or palliative to a local tissue at the point of introduction. In some embodiments, the suppository is a carrier of a therapeutic agent for systemic and/or local action.

In one embodiment, a suppository is a rectal suppository, a vaginal suppository, or urethral suppository.

Non-limiting examples of carriers for pharmaceutical compositions being in the form of a suppository include but are not limited to: cocoa butter (relates to a mixture of triglycerides of saturated and unsaturated fatty acids, such as stearic, palmitic, oleic, lauric, and linoleic acids), a cocoa butter substitute (relates to vegetable oils modified by esterification, hydrogenation, etc., including hydrogenated vegetable oil and hard fat), glycerinated gelatin, a polyethylene glycol-based carrier, and a surfactant (such as polyoxyethylene sorbitan fatty-acid esters and polyoxyethylene stearates) or any combination thereof.

Non-limiting examples of carriers for pharmaceutical compositions being in the form of a cream include but are not limited to: non-ionic surfactants (e.g., glyceryl monolinoleate glyceryl monooleate, glyceryl monostearate lanolin alcohols, lecithin mono- and di-glycerides poloxamer polyoxyethylene 50 stearate, and sorbitan trioleate stearic acid), anionic surfactants (e.g. pharmaceutically acceptable salts of fatty acids such as stearic, oleic, palmitic, and lauric acids), cationic surfactants (e.g. pharmaceutically acceptable quaternary ammonium salts such as benzalkonium chloride, benzethonium chloride, and cetylpyridinium chloride) or any combination thereof.

In some embodiments, the pharmaceutical composition being in the form of a cream further comprises a thickener.

Non-limiting examples of thickeners include, but are not limited to microcrystalline cellulose, a starch, a modified starch, gum tragacanth, gelatin, and a polymeric thickener (e.g. polyvinylpyrrolidone) or any combination thereof.

In some embodiments, the pharmaceutical composition further comprises a pharmaceutical agent such as an anti-inflammatory agent, an analgesic, an antimicrobial agent or any combination thereof.

Non-limiting examples of analgesics include but are not limited to: methyl salicylate, menthol, camphor, eucalyptol, capsicum, ibuprofen, aspirin, paracetamol, and rofecoxib or any pharmaceutically acceptable salts or mixtures thereof.

Non-limiting examples of anti-inflammatory agents include but are not limited to: non-steroidal anti-inflammatory agents such as celecoxib, and etoricoxib, diclofenac, fenoprofen, flurbiprofen, ketoprofen, naproxen, etodolac, and diflunisal or any pharmaceutically acceptable salts or mixtures thereof.

Non-limiting examples of antimicrobial agents include but are not limited to: beta-lactam antibiotics, aminoglycoside antibiotics, tetracycline antibiotics, trimethoprim antibiotics, nitrofurantoin antibiotics and pharmaceutically acceptable salts thereof, and mixtures thereof.

In some embodiments, the pharmaceutical composition further comprises an additive. Non-limiting examples of additives include, but are not limited to: a thickener, a filler, a colorant, and an excipient or any combination thereof.

In some embodiments, the pharmaceutical composition is in a form of an enema composition. In some embodiments, an enema composition is an aqueous composition. In some embodiments, an aqueous composition is in a form of a solution, a dispersion, or a suspension.

In some embodiments, the enema composition comprises an aqueous solvent as a carrier. Non-limiting examples of aqueous solvents include but are not limited to: an aqueous salt solution, an aqueous buffer solution, saline, and water or any combination thereof.

In some embodiments, the pH of the enema composition is at a range from 5 to 8, from 5.5 to 7.5, from 6 to 7.5, from 6.5 to 7, from 7 to 8, from 5.5 to 6, from 5 to 6.5 including any range or value therebetween. Each possibility represents a separate embodiment of the invention.

In some embodiments, the carrier of the enema composition further comprises any a surfactant. In some embodiments, a surfactant comprises any of a non-ionic surfactant, a cationic surfactant, an anionic surfactant as described hereinabove.

In some embodiments, the enema composition further comprises an antioxidant, a preservative or both, wherein the antioxidant and the preservative are as described hereinabove. In some embodiments, the enema composition further comprises an agent for the adjustment of tonicity, as described hereinabove. In some embodiments, the enema composition further comprises an anti-foaming agent.

In some embodiments, the pharmaceutical composition comprises the cannabinoid (e.g., CBD-C5) and the HA component, presented in a unit dosage form and prepared by any of the methods well known in the art of pharmacy. In one embodiment, the unit dosage form is in the form of a suppository, patch, or a pre-filled container.

In one embodiment, the present invention provides combined preparations. In one embodiment, “a combined preparation” defines especially a “kit of parts” in the sense that the combination partners as defined above can be dosed independently or by use of different fixed combinations with distinguished amounts of the combination partners i.e., simultaneously, concurrently, separately or sequentially. In some embodiments, the parts of the kit of parts can then, e.g., be administered simultaneously or chronologically staggered, that is at different time points and with equal or different time intervals for any part of the kit of parts. The ratio of the total amounts of the combination partners, in some embodiments, can be administered in the combined preparation. In one embodiment, the combined preparation can be varied, e.g., in order to cope with the needs of a patient subpopulation to be treated or the needs of the single patient which different needs can be due to a particular disease, severity of a disease, age, sex, or body weight as can be readily made by a person skilled in the art.

In another aspect, there is a kit comprising a first compartment comprising the cannabinoid and a second compartment comprising the HA component and/or the SCF, as disclosed herein. In some embodiments, the first compartment and the second compartment each independently comprise a pharmaceutically effective amount of the active ingredient (cannabinoid, SCF, and/or HA component). In some embodiments, the first compartment and the second compartment are further packaged in a container (e.g. a sealed and/or an airtight container or package). In some embodiments, compositions of the present invention are presented in a pack or dispenser device, such as an FDA approved kit, which contains, one or more unit dosage forms containing the active ingredients. In one embodiment, the pack, for example, comprises metal or plastic foil, such as a blister pack.

In some embodiments, the first and/or the second compartment of the kit is in a unit dosage form, wherein each unit dosage form comprises a therapeutically effective amount of the active ingredient (e.g. dosage form as described herein). In some embodiments, the first compartment and the second compartment of the kit are formulated for simultaneous, or subsequent administration.

In some embodiments, the kit further comprises instructions for subsequent or for simultaneous administration of the first and of the second compartment of the kit. In some embodiments, the kit optionally comprises instructions for mixing the first and the second compartment of the kit at a predefined ratio prior to administration (optionally wherein the ratio is a w/w ratio or a molar ratio disclosed herein). In some embodiments, the instructions comprise guidelines with respect to dosing, ratio of first and the second compartment, and a suitable administration regimen.

In some embodiments, provided herein a combination comprising a therapeutically effective amount of the active ingredients (e.g., the cannabinoid, and the HA component) and an acceptable carrier for use in the treatment or prevention of a disorder. In some embodiments, the combination comprises the cannabinoid formulated within a first pharmaceutical composition and the HA component formulated within a second pharmaceutical composition. In some embodiments, the combination comprises a therapeutically effective amount of CBD-C5 formulated within a first pharmaceutical composition and a therapeutically effective amount of the HA component formulated within a second pharmaceutical composition, wherein the first pharmaceutical composition and the second pharmaceutical composition are as described hereinabove.

In some embodiments, the combination of the invention is as exemplified in the Examples section.

### Composition for Use

In some embodiments, provided herein the pharmaceutical composition of the invention for use in the prevention or treatment of a disease or a disorder associated with rectal and/or intestinal inflammation. In some embodiments, the pharmaceutical composition comprises an effective amount (e.g. dosage form, as disclosed herein) of the active ingredients of the composition of the invention.

In some embodiments, the disorder is associated with an inflammation of the ileal tissue. In some embodiments, the disorder is associated with an inflammation of the duodenum. In some embodiments, the disorder is associated with an inflammation of the jejunum. In some embodiments, the disorder is associated with an inflammation of the distal ileum, inflammation of the terminal ileum or any combination thereof.

In some embodiments, the disorder is selected from the group comprising: pouchitis, inflammatory bowel disease (e.g., Crohn's disease and ulcerative colitis) or any combination thereof.

In some embodiments, the pharmaceutical composition as described herein comprises the combination described herein. In some embodiments, the pharmaceutical composition or the combination of the invention is for use in the prevention or treatment of pouchitis.

In some embodiments, there is provided a pharmaceutical composition or combination comprising an effective amount of the cannabinoid (e.g., CBD-C5), for use in enhancing the therapeutic efficacy of the HA component. In some embodiments, there is provided a pharmaceutical composition or combination comprising an effective amount of the HA component, for use in enhancing the therapeutic efficacy of the cannabinoid (e.g., CBD-C5).

In another embodiment, measures (e.g., dosing and selection of the complementary agent) are taken to adverse side effects which are associated with combination therapies.

In one embodiment, depending on the severity and responsiveness of the condition to be treated, dosing can be of a single or a plurality of administrations, with course of treatment lasting from several days to several weeks or until cure is affected or diminution of the disease state is achieved.

In some embodiments, the composition of the present invention is administered in a therapeutically safe and effective amount. As used herein, the term “safe and effective amount” refers to the quantity of a component which is sufficient to yield a desired therapeutic response without undue adverse side effects, including but not limited to toxicity, such as calcemic toxicity, irritation, or allergic response, commensurate with a reasonable benefit/risk ratio when used in the presently described manner. The actual amount administered, and the rate and time-course of administration, will depend on the nature and severity of the condition being treated. Prescription of treatment, e.g. decisions on dosage, timing, etc., is within the responsibility of general practitioners or specialists, and typically takes account of the disorder to be treated, the condition of the individual patient, the site of delivery, the method of administration and other factors known to practitioners. Examples of techniques and protocols can be found in Remington: The Science and Practice of Pharmacy, 21st Ed., Lippincott Williams & Wilkins, Philadelphia, Pa., (2005).

In some embodiments, the effective amount or dose of the active ingredient can be estimated initially from in vitro assays. In one embodiment, a dose can be formulated in animal models and such information can be used to more accurately determine useful doses in humans.

In one embodiment, toxicity and therapeutic efficacy of the active ingredients described herein can be determined by standard pharmaceutical procedures in vitro, in cell cultures or experimental animals. In one embodiment, the data obtained from these in vitro and cell culture assays and animal studies can be used in formulating a range of dosage for use in human. In one embodiment, the dosages may vary depending on the dosage form employed and the route of administration utilized. In one embodiment, the exact formulation, route of administration and dosage can be chosen by the individual physician in view of the patient's condition. [See e.g., Goodman and Gilman's The Pharmacological Basis of Therapeutics, 13th Ed., McGraw-Hill/Education, New York, NY (2017)].

In some embodiments, pharmaceutical compositions containing or comprising the cannabinoid (e.g., CBD-C5) and the HA component as active ingredients, can be prepared according to conventional pharmaceutical compounding techniques. See, for example, Remington: The Science and Practice of Pharmacy, 22nd Ed., Pharmaceutical Press, Philadelphia, PA (2012).

In some embodiments, compositions including the preparation of the present invention formulated in a compatible pharmaceutical carrier are prepared, placed in an appropriate container, and labeled for the treatment of an indicated condition.

In some embodiments, compositions of the present invention are presented in a pack or dispenser device, such as an FDA approved kit, which contains, one or more unit dosage forms containing the active ingredients. In one embodiment, the pack, for example, comprises metal or plastic foil, such as a blister pack. In one embodiment, the pack or dispenser device is accompanied by instructions for administration. In one embodiment, the pack or dispenser is accommodated by a notice associated with the container in a form prescribed by a governmental agency regulating the manufacture, use or sale of pharmaceuticals, which notice is reflective of approval by the agency of the form of the compositions or human or veterinary administration. Such notice, in one embodiment, is labeling approved by the U.S. Food and Drug Administration for prescription drugs or of an approved product insert.

### Methods

According to some embodiments, there is provided a method for preventing or treating a disease or disorder associated with rectal and/or intestinal inflammation in a subject in need thereof, comprising administering to a subject a therapeutically effective amount of the composition or the combination (e.g. kit) of the invention. In some embodiments, preventing or treating comprises reducing or ameliorating at least one symptom associated with the disease or disorder disclosed herein. In some embodiments, the therapeutically effective amount is sufficient for at least 10%, at least 20%, at least 30%, at least 50%, at least 70%, at least 80%, at least 90%, or a complete reduction at least one symptom associated with the disease or disorder disclosed herein, including any range between. In some embodiments, the therapeutically effective amount is or comprises the dosage form as disclosed herein.

In some embodiments, the method comprises administering to a subject a pharmaceutical composition comprising CBD and the HA component as active ingredients, wherein the CBD and the HA component are as described hereinabove. In some embodiments, the method comprises administering to the subject a pharmaceutical composition or combination of the CBD-C5 and the HA component.

In some embodiments, the method for preventing or treating a disorder associated with rectal and/or intestinal inflammation comprises administering to the subject a pharmaceutical composition comprising: (i) CBD-C5 at a w/w concentration from 0.1 to 50%, and (ii) the HA component at a w/w concentration from 1 to 90%.

In some embodiments, the method for preventing or treating a disorder associated with rectal and/or intestinal inflammation comprises administering to the subject a therapeutically effective amount of the pharmaceutical composition comprising: (i) CBD-05 at a w/w concentration from 0.1 to 50%, (ii) the HA component at a w/w concentration from 1 to 90%, and (iii) an additional compound comprising SCF (e.g. butyric acid, and/or propionic acid including any salt thereof), inulin or both at a w/w concentration from 0.1 to 50%.

In some embodiments, the therapeutically effective amount is or comprises between 5 and 50 mg, between 5 and 10 mg, between 5 and 20 mg, between 10 and 20 mg, between 20 and 50 mg, between 10 and 50 mg, between 20 and 30 mg, between 10 and 30 mg, including any range between of the cannabinoid (e.g. CBD-C5) per kg body weight of the subject; and between 30 and 300 mg, between 30 and 100 mg, between 30 and 50 mg, between 50 and 100 mg, between 50 and 300 mg, between 50 and 200 mg, between 100 and 300 mg, between 200 and 300 mg, including any range between of the SCF (e.g. propionic acid and/or butyric acid and/or any combination thereof) and/or a salt thereof per kg body weight of the subject. In some embodiments, each dosage form (e.g. enema) further comprises HA (e.g. as an aqueous HA solution) at a concentration of about 2% w/w.

In some embodiments, the method comprises administering CBD (e.g. as part of the kit or of the composition) in a daily dose of at least 5 mg, at least 15 mg, at least 50 mg, at least 100 mg, at least 200 mg, at least 300 mg, at least 400 mg, at least 500 mg, or at least 600 mg, at least 750 mg, at least 1,000 mg, or at least 2000 mg, or any value and range therebetween. Each possibility represents a separate embodiment of the invention. In some embodiments, CBD is administered in a daily dose of to the subject at a dosage of 5-50 mg, 10-100 mg, 10-250 mg, 50-350 mg, 100-700 mg, 5-500 mg, 220-450 mg, 70-400 mg, 1-350 mg, 30-490 mg, 50-550 mg, 150-850 mg, 250-1,000 mg, 1000-2000 mg, or 400-1,250 mg. Each possibility represents a separate embodiment of the invention.

In some embodiments, the method comprises administering the HA component in a daily dose of at least 5 mg, at least 15 mg, at least 50 mg, at least 100 mg, at least 200 mg, at least 300 mg, at least 400 mg, at least 500 mg, or at least 600 mg, at least 750 mg, at least 1,000 mg, at least 1200 mg, at least 5 g, at least 10 g, at least 20 g, at least 30 g, at least 40 g, at least 50 g, at least 60 g, at least 70 g, at least 80 g, or at least 100 g, including any value and range therebetween. Each possibility represents a separate embodiment of the invention. In some embodiments, the HA component is administered in a daily dose ranging from 5-50 mg, 10-100 mg, 10-250 mg, 50-350 mg, 100-700 mg, 5-500 mg, 220-450 mg, 70-400 mg, 1-350 mg, 30-490 mg, 50-550 mg, 150-850 mg, 250-1,000 mg, 400-1,250 mg, 2-5 g, 5-10 g, 10-20 g, 20-40 g, 40-50 g, 50-60 g, 60-70 g, 70-80 g, or 80-100 g including any value and range therebetween. Each possibility represents a separate embodiment of the invention.

In some embodiments, the method further comprises administering the additional compound comprising SCF (e.g. butyric acid, and/or propionic acid including any salt thereof), inulin or both in a daily dose of at least 5 mg, at least 15 mg, at least 50 mg, at least 100 mg, at least 200 mg, at least 300 mg, at least 400 mg, at least 500 mg, at least 1000 mg, at least 2000 mg, at least 3000 mg, at least 5 g, at least 10 g, at least 20 g, at least 30 g, at least 40 g, at least 50 g, at least 60 g, at least 70 g, at least 80 g, or at least 100 g, including any value and range therebetween. Each possibility represents a separate embodiment of the invention. In some embodiments, the HA component is administered in a daily dose ranging from 5-50 mg, 10-100 mg, 100-700 mg, 700-1,000 mg, 2-5 g, 5-10 g, 10-20 g, 20-40 g, 40-50 g, 50-60 g, 60-70 g, 70-80 g, or 80-100 g including any value and range therebetween. Each possibility represents a separate embodiment of the invention.

In some embodiments, CBD, the HA component and optionally the additional compound are administered simultaneously. In some embodiments, CBD, the HA component and optionally the additional compound are administered consecutively. In some embodiments, CBD, the HA component and optionally the additional compound are administered sequentially.

In some embodiments, the method comprises a topical administration, a rectal administration or both. For rectal, vaginal, urethral and/or topical applications, the pharmaceutical composition is in the form of, for example, and not by way of limitation, an ointment, a cream, a gel, a paste, a foam, a suppository, a pad or a gelled stick.

In some embodiments, the method comprises administering a therapeutically effective amount of the pharmaceutical composition or the combination of the invention at least 1 time, at least 2 times, at least 3 times, at least 4 times, at least 5 times, at least 7 times, or at least 10 times per day, or any value and range therebetween. Each possibility represents a separate embodiment of the invention. In some embodiments, the method comprises administering the composition or the combination of the invention 1-2 times per day, 1-3 times per day, 1-4 times per day, 1-5 times per day, 1-7 times per day, 2-3 times per day, 2-4 times per day, 2-5 times per day, 3-4 times per day, 3-5 times per day, or 5-7 times per day. Each possibility represents a separate embodiment of the invention. In some embodiments, therapeutically effective amount of the pharmaceutical composition or the combination of the invention is or comprises the dosage form disclosed herein.

In some embodiments, the subject is afflicted with a rectal and/or intestinal inflammatory disease. In some embodiments, a rectal and/or intestinal inflammatory disease comprises one or more diseases or conditions selected from: pouchitis, inflammation of the distal ileum, inflammation of the terminal ileum, inflammatory bowel disease (e.g., Crohn's disease and ulcerative colitis), ulcerative colitis, indeterminate colitis, diversion colitis, distal colitis, proctitis, proctosigmoiditis, left-sided colitis, extensive colitis, pancolitis, internal hemorrhoids, external hemorrhoids, rectal bleeding, and familial adenomatous polyposis (FAP) or any combination thereof. In some embodiments, the subject has undergone a colectomy.

In some embodiments, the subject is a mammal. In some embodiments, the subject is a lab animal. In some embodiments, the subject is a pet. In some embodiments, the subject is a rodent. In some embodiments, the subject is a farm animal. In some embodiments, the subject is a human subject.

### General

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life.

As used herein, the term “prevention” of a disease, disorder, or condition encompasses the delay, prevention, suppression, or inhibition of the onset of a disease, disorder, or condition. As used in accordance with the presently described subject matter, the term “prevention” relates to a process of prophylaxis in which a subject is exposed to the presently described active ingredients prior to the induction or onset of the disease/disorder process. This could be done where an individual has a genetic pedigree indicating a predisposition toward occurrence of the disease/disorder to be prevented. For example, this might be true of an individual whose ancestors show a predisposition toward certain types of inflammatory disorders.

The term “suppression” is used to describe a condition wherein the disease/disorder process has already begun but obvious symptoms of the condition have yet to be realized. Thus, the cells of an individual may have the disease/disorder, but no outside signs of the disease/disorder have yet been clinically recognized. In either case, the term prophylaxis can be applied to encompass both prevention and suppression.

Conversely, the term “treatment” refers to the clinical application of active agents to combat an already existing condition whose clinical presentation has already been realized in a patient.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a”, “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise”, “include”, and “have” and conjugates thereof, are used to indicate that the object or objects of the verb are not necessarily a complete listing of components, elements or parts of the subject or subjects of the verb.

Other terms as used herein are meant to be defined by their well-known meanings in the art.

Unless specifically stated or obvious from context, as used herein, the term “or” is understood to be inclusive.

Throughout this specification and claims, the word “comprise” or variations such as “comprises” or “comprising” indicate the inclusion of any recited integer or group of integers but not the exclusion of any other integer or group of integers.

In some embodiments, as used herein, the term “consists essentially of” or variations such as “consist essentially of” or “consisting essentially of” as used throughout the specification and claims, indicate the inclusion of any recited compounds, and the optional inclusion of any recited compound(s) that do not materially change the basic or novel properties of the specified method, structure or composition. In some embodiments, the term “consisting essentially of” is used to define formulations which include the recited elements as the only active ingredients thereof, but exclude other elements that may have an essential significance on the formulation.

As used herein, the terms “comprises”, “comprising”, “containing”, “having” and the like can mean “includes”, “including”, and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments. In one embodiment, the terms “comprises” “comprising”, and “having” are/is interchangeable with “consisting”.

### EXAMPLES

Generally, the nomenclature used herein, and the laboratory procedures utilized in the present invention include molecular, biochemical, and microbiological techniques. Such techniques are thoroughly explained in the literature.

### Materials and Methods

### Cells Culture and Treatments

Cell lines were cultured according to standard mammalian tissue culture protocols and sterile technique. Human colon epithelial cells, Caco-2, were cultured in high glucose Dulbecco's Modified Eagle Medium (DMEM). Human monocytes THP-1 cells line were cultured in RPMI medium 1640. Both media were supplemented with 10% fetal bovine serum (FBS), 2 mM 1-glutamine and 0.1% penicillin-streptomycin solution in a humidified atmosphere (5% CO2, 95% air, 37° C.) in a 75 cm2 cell culture treated flask (Eppendorf). All the media and supplements were obtained from Biological Industries.

Treatment was performed by plating cells at a starting confluence of 2×10⁴ cells/well in a 96 micro well delta surface plates (Eppendorf) or 6×10⁴ cells/well in a 6.5 mm Transwell with a 0.4 μm pore polycarbonate membrane insert (Millipore®).

### MTT Viability Assay

The viability of the cells following treatment was determined using a commercially available MTT assay kit (Abcam, ab146345) and performed according to manufacturer's instructions. Briefly, cells were seeded in a 96-well plate at a density of 2×104 cells/well (n=4) in 100 μl cell specific media. After overnight incubation, cells were exposed the samples for 24 h incubation. According to the MTT standard protocol, after 24 h treatment, the media was removed, and all cells were incubated with serum-free media containing 0.5 mg/ml MTT for 4 hours at the incubator. The MTT purple crystals formed by the viable cells were dissolved using isopropanol containing 0.04 mol/L HCl. The quantification was determined by measuring the optical density at 570 nm in a spectrophotometer reader (Spark, Tecan). Results were presented as proportional viability (%) by comparing between treated and untreated groups.

Cytotoxic potential of an extract was determined according to the following criteria:
Cytotoxic: Viability is reduced to less than 70% of vehicle control.
Non-cytotoxic: Viability is >70% of vehicle control.

Interleukin 8 (IL 8) detection and quantification was performed with Homogeneous Time-Resolved fluorescence (HTRF).

Caco-2 cells were seeded in a 96 micro well delta surface plates (Eppendorf) at a starting confluence of 2×10⁴ cells/well and were incubated for 21 days. Growth medium was replaced every 2-3 days.

After 22 days, the medium was replaced with medium containing both TNFα (as a damage inducer) and the tested sample (as treatment) using specific media containing 5% FBS. After 24 hours incubation, the supernatant was collected and stored for cytokine analysis. The supernatant was diluted up to 5 times in working media to avoid the hook effect.

Human THP-1 monocytes were seeded in a 96-well plate (Eppendorf) at a density of 3×10⁵ cells/well (n=4) in 100 μl cell specific media and were differentiated using phorbol-12-myristate 13-acetate (PMA) (Sigma-Aldrich) at a final concentration of 200 nM. PMA supplemented media was removed after 72 hours. Cells were then washed twice with PBS and rested in fresh PMA-free media for further 24 hours in order to obtain phenotypic characteristics of macrophages (Daigneault et al., 2010). After 24 hours, cells were stimulated by Lipopolysaccharides (LPS) at a final concentration of 1 μg/ml and were treated concomitantly with the samples using specific media containing 5% FBS. After 24 h, the cells supernatants were harvest and diluted up to 5 times in working media to avoid the hook effect.

HTRF assays were performed in white 96-well plate (CisBio Bioassays) with a total working volume of 20 μL. All HTRF reagents were purchased from CisBio Bioassays and reconstituted according to the manufacturer protocols. For each assay 16 μL of diluted supernatants samples were incubate with 4 μL mixed solution containing donor and acceptor antibodies.

After 2 h incubation HTRF signals were measured using SPARK multimode microplate reader with an excitation filter at 320 nm and fluorescence wavelength measurement at 620 and 665 nm, an integration delay of 60 μs and an integration time of 400 μs. Results were analyzed with a two-wavelength signal ratio: [intensity (665 nm)/intensity (620 nm)]*104. Calculation and analysis of cytokines release was performed according to the manufacturer protocols and using the 4PL 1/Y2 formula in GraphPad Prism software.

### Cytokine-Induced Epithelial Damage Transepithelial Electrical Resistance (TEER) Measurement

For TEER measurement, Caco-2 cells were seeded in 6.5 mm Trans-well with a 0.4 μm pore polycarbonate membrane insert (Millipore) at a seeding density of 6×10⁴ cells/well.

The culture medium was added to both apical and the basal compartments. Medium was changed every 3-4 days for 21 days. Measurement of TEER was performed before and after TNFα challenge to evaluate damage of the cellular monolayer during the challenge and the ability of CBD and or bacterial metabolites to protect the monolayer. The values of TEER were determined by measuring the potential difference between the two sides of the cell monolayer at all three different points at the edges of the filter using a EVOM2 voltammeter (Millicell) connected to a pair of chopstick electrodes. Trans-well filters showing TEER values<1500 ohm were excluded from this study.

On the day of experiments, the cells were washed twice with pre-warmed medium.

After a baseline TEER measurement, the Caco-2 cell monolayers were insulted by TNFα (400 ng/ml) on the basal side of the trans-well chamber and were concomitantly treated with either CBD, sodium butyrate (NaB), sodium propionate (NaP) or sample combinations on the apical side of the chamber. TEER measurement was performed at 24, 48 and 72 hours after treatment.

### RNA Purification and qRT-PCR Analysis of Gene Expression

RNA was purified from every trans-well triplicate immediately after final TEER measurement, using RNeasy mini kit (QIAGEN), according to manufacturer instructions. RNA concentration and purity were determined by using the UV-Vis micro volume Nanodrop lite (Thermo scientific). cDNA was synthesized from the purified RNA using the High-Capacity reverse transcription kit (Thermo scientific, USA). qRT-PCR reaction was preformed using a Fast SYBR Green Master Mix (Thermofisher, USA) in The StepOnePlus Real-Time PCR System (Applied Biosystems). The cycle conditions for real-time PCR were 95° C. for 10 min, followed by 40 cycles of 95° C. for 15 sec, and 60° C. for 1 min. The experiment included three repeats wherein each repeat was in triplicate.

TABLE 1
summarizes the primers used in this study.

| Target | Forward primer | Reverse primer |
|---|---|---|
| GAPDH | TGTAGTTGAGGTCAATGA | ACATCGCTCAGACACCATG |
| | AGGG (SEQ ID NO: 1) | (SEQ ID NO: 2) |
| ZO1 | GCTGGCTTATTCTGAGAT | CGCGTCTCTCCACATACAT |
| | GGA (SEQ ID NO: 3) | TC (SEQ ID NO: 4) |
| Ki-I67 | GAAGCTGGATACGGATGT | CGCCTGGTTACTATCAAAA |
| | CA (SEQ ID NO: 5) | GGA (SEQ ID NO: 6) |
| CASP3 | GTTTGCTGCATCGACATC | CTCTGGAATATCCCTGGAC |
| | TG (SEQ ID NO: 7) | AAC (SEQ ID NO: 8) |

### EXAMPLE 1

### IL8 Secretion from LPS-Activated THP-1 Cells

Various concentrations of CBD, sodium propionate (NaP) and sodium butyrate (NaB), were compared to a combination of CBD and NaP or of CBD and NaB respectively. The tested compositions have been incubated with LPS-stimulated THP-1 Cells, as described.

Two different combinations of the substances were prepared in order to evaluate possible synergistic effect. Combo 1 included CBD (30 μM) and NaB (20 mM) and Combo 2 included CBD (30 μM) and NaP (20 mM).

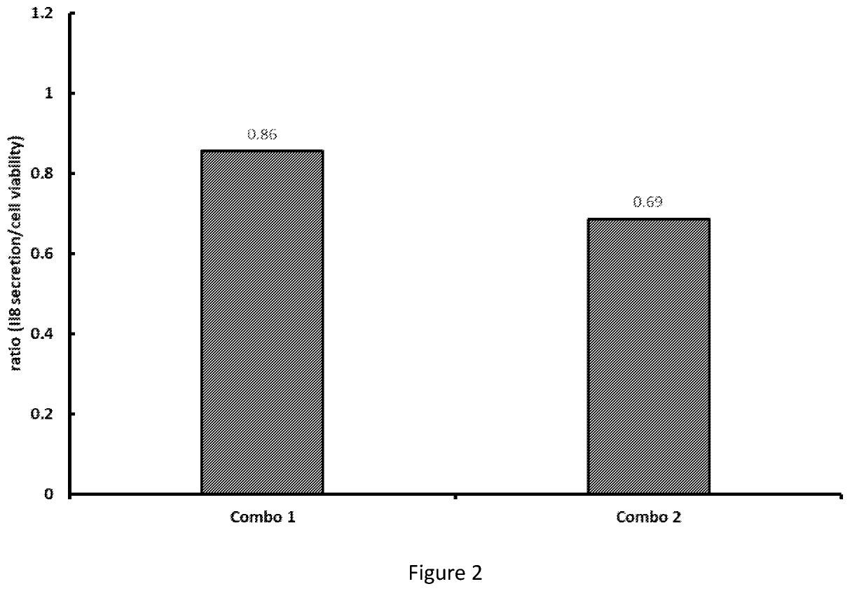

In order to evaluate a possible synergistic effect on IL8 secretion from LPS-stimulated THP-1 cells, LPS-stimulated THP-1 cells were incubated with Combo 1 or with Combo 2, so that the final concentration of (i) CBD, and of (ii) NaB or NaP in the incubation medium was 30 μM and 20 mM, respectively. As shown in FIG. 2, Combo 1 produced a weak effect (ratio of 0.86), while Combo 2 produced a substantial effect (ratio of 0.69), thus pointing out superior biological effect of the combination of NaP with CBD.

### EXAMPLE 2

### IL8 Secretion from TNFA Induced CACO2 Cells

The tested composition did not exhibit a significant effect on CACO2 cell viability. It was observed that only NaP was able to induce mild reduction of IL8 secretion from TNFα induced Caco2 cells, in contrast to CBD and NaB alone, which didn't exhibit any significant effect.

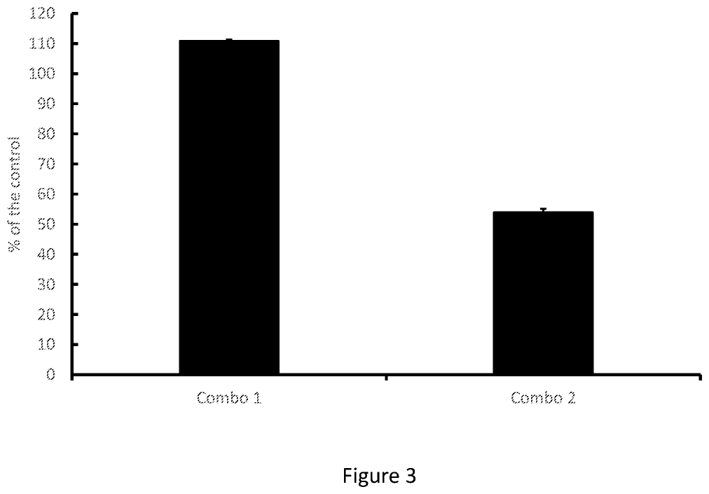

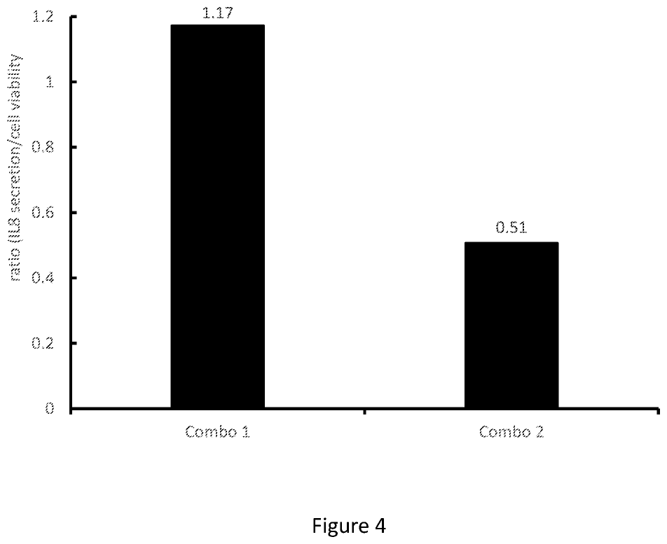

Synergistic effect of the combinations resulting in a significant reduction in IL8 secretion levels as compared to the untreated isolates has been demonstrated in this specific model (FIGS. 3 and 4). Combo 2, which is composed of CBD and NaP, reduced IL8 secretion levels to a greater extent than Combo 1.

### EXAMPLE 3

### In-Vivo Experiments

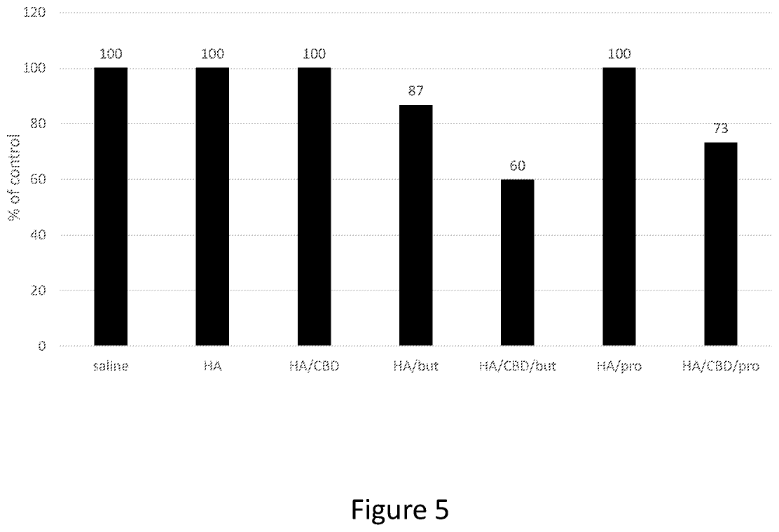

In order to assess treatment efficacy, exemplary compositions of the invention have been tested in-vivo. The treatment efficacy of the compositions disclosed herein has been determined and scored based on the attenuation of ulcerative colitis-like symptoms in the induced-colitis DSS mouse model. The results of the in-vivo studies are exemplified by FIGS. 5 to 7.

### Materials and Methods

The DSS model is one of the leading models for the assessment of treatments on the integrity of the colons and is known to produce clinical and histopathological changes mimicking some of the features seen in the clinical IBD. It includes induction of inflammation via continuous administration of DSS over the course of 5 days. DSS model is a chemically induced study of Inflammatory Bowel Disease (IBD), using dextran sodium sulphate (DSS) in drinking water that induces ulcerative colitis-like inflammation. The disease is descending colon specific characterized by gross bleeding, thickened colon walls with occasional adhesions, pseudo polyps, redness and oedema, as well as reduced overall colon length.

42 Female c57blk Mice were used for this study at the age of 7-8 weeks:
Weight: 19-25 gr at initiation of experiment; acclimatization period: 11 days, to allow animals reach the right weight of nearly 22 g on average; identification: a cage card contained the study name, animal number and relevant details such as treatment group. The mice are numbered with ear tag. All Forty-two (42) females of c57blk mice were induced with ulcerative colitis using Dextran Sulphate Sodium (DSS)

### Formulations and Treatment

Seven (7) groups of animals (n=6, per groups) were treated with DSS. On the fourth day, animals were subjected to rectal treatment with different formulations; group 1—saline, group 2—Hyaluronic Acid (HA), group 3—HA+CBD, group 4, HA+sodium butyrate, group 5-sodium-propionate, group 6-HA+Na—B+CBD, and group 7-HA+Na—P+CBD.

Clinical examination to assess the progress of disease with the different treatments were performed including disease index score (DAI), body weight, occult blood, and feces consistency. At termination, excised colons' weight and length were assessed, as well as histopathology.

The tested composition was prepared as formulation for rectal administration (locally to the colon). The formulations contained hyaluronic acid (HA) as an inactive ingredient, yet it might improve absorption of the active ingredients into the tissue. Seven (7) treatment formulations were prepared, designated for the treatment of 7 groups, as follows:
Sodium Butyrate (NaB) in HA: sodium butyrate in HA at a concentration of 1 mg/mL to give a dose of 20 mg/kg.
Sodium propionate (NaP) in HA: sodium propionate in HA at a concentration of 0.5 mg/mL to give a dose of 10 mg/kg.
Cannabidiol (CBD) (synthetic form) in HA: CBD emulsified in HA at a concentration of 1 mg/mL to give a dose of 20 mg/kg.
HA+NaB+CBD: Both NaB and CBD are emulsified in HA at the a concentration of the 1 mg/mL to give a dose of 20 mg/kg for each of the test items.
HA+NaP+CBD: both NaP and CBD are emulsified in HA at a concentration of 0.5 and 1 mg/mL, respectively, to give a dose of 10 and 20 for NaP and CBD, respectively.
HA and saline were administered as received (without any additional excipients).

The test animals were divided into 6 groups: group 1 and 2 (negative controls) were treated with saline and HA, respectively; group 4 and 5 were treated with a combination of HA with butyrate and propionate salt (SCF), respectively; group 6 and were treated with exemplary compositions of the invention, as represented in Table 2 below.

TABLE 2

| Group # | Treatment | Dose (mg/kg) | | | Volume | Route of | Treatment |
|---|---|---|---|---|---|---|---|
| (n = 6/grp) | via rectus | CBD | NaB | NaP | (μl) | Administration | regimen |
| Group 1 | Saline | | | | 100 | Rectally | Starting |
| Group 2 | HA | | | | 100 | (enema) | D3-D10 |
| Group 3 | HA + CBD | 15 | | | 100 | | |
| Group 4 | HA + Na- | | 100 | | 100 | | |
| | Butyrate | | | | | | |
| Group 5 | HA + Na- | | | 75 | 100 | | |
| | Propionate | | | | | | |
| Group 6 | HA + Na- | 15 | 100 | | 100 | | |
| | Butyrate + | | | | | | |
| | CBD | | | | | | |
| Group 7 | HA + Na- | 15 | | 75 | 100 | | |
| | Propionate + | | | | | | |
| | CBD | | | | | | |

For untreated mice, remarkable lesions were found expressed by mucosal ulceration, necrosis, and inflammation. The lesions were very clear and were composed of loss and necrosis of the mucosal epithelium and an inflammatory reaction. In the severe cases more involvement of neutrophils was shown.

The severity of the histopathological findings in the different groups, obtained by the semi-quantitative analysis is described herein. The control mice treated by saline, and HA as alone treatment (groups 1 and 2), had prominent lesions (grade 8.67). Severe ulceration of large parts of the mucosa, erosion, and crypt loss with many cellular debris in the gut lumen. Inflammatory reactions were seen around and within the lesions. Mice treated with HA and CBD (group 3), also showed similar results as obtained in the control groups.

The severity of the lesions in group 4 treated with HA and sodium butyrate, was lower than the control groups but not statistically significant. Groups 6 and 7 which include CBD and SCF as active ingredients, exhibited the lowest severity score (grade 5.17) as compared to the group 4 (grade 7.5) and group 3 (grade 8.7). The synergistic compositions of the invention with a synergy score of about 0.68, showed a significant reduction in pathological score when compared to control group (P<0.05).

As presented by FIG. 5, exemplary compositions of the invention including CBD and an SCF (e.g. sodium butyrate or sodium propionate), exhibited a synergistic effect and significantly reduced the disease severity (by about 40% and about 30%, respectively), compared to the untreated mice (saline), HA and to a combined treatment with HA and sodium propionate. Noteworthy, a combined treatment with HA and sodium butyrate showed a minimal improvement (about 13% reduction), however the exemplary compositions exhibited significantly greater reduction of the inflammation, as reflected by the histopathology index.

An increase in the DAI of the DSS mice in all groups induced with ulcerative colitis was initially seen on day 3. The DAI score for all groups was similar prior to treatment, on day 4. On day 6 an apparent peak score for the DAI was reached for all groups, representing acute disease.

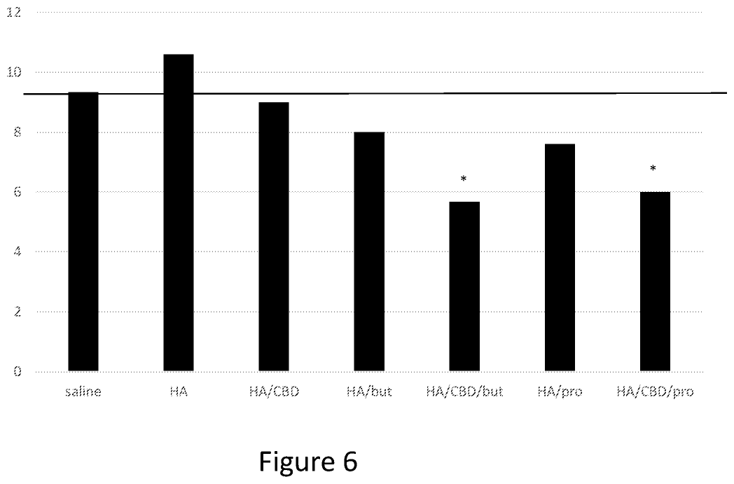

The DAI scores of the different groups on day 6 are represented by FIG. 6. The control groups 2 and 3, showed an increase in the DAI score, reaching peak levels score about 10, which was maintained for the duration of the study, indicating that the DSS treatment induced ulcerative colitis in the test animals.

Groups 6 and 7 treated with the exemplary compositions of the invention (HA+CBD+butyrate and HA+CBD+propionate) showed significant (p<0.05) decrease (about 50% reduction) of the DAI score (DAI of 5.8 and 6, respectively), as compared to the control group.

In addition, a synergy between CBD and butyrate and/or CBD and propionate was demonstrated as compared to the alone treatments. The synergy score of the compositions of the invention was about 0.7, whereas treatments with HA and CBD and/or with HA and propionate didn't exhibit any substantial synergism (synergy score of 0.92).

Furthermore, the excised colons of the treated mice were measured for their weight and length, an indicative severity sign of the induced colitis.

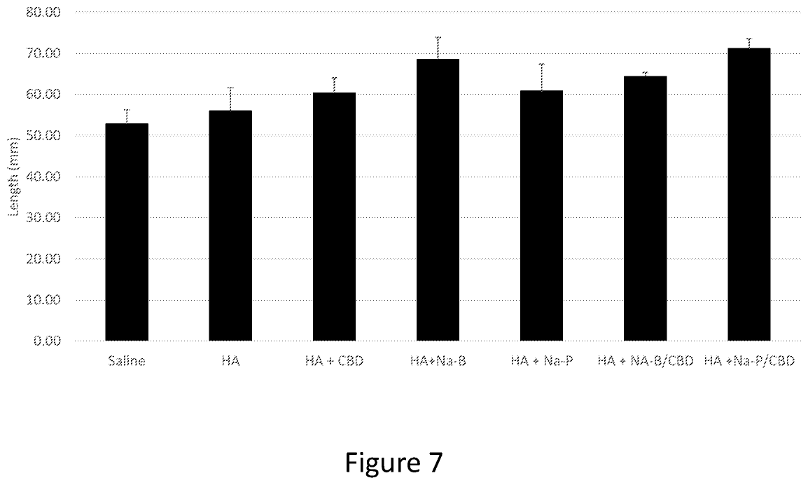

Additional synergistic effects of the exemplary compositions of the invention is further demonstrated in FIG. 7, showing significant increase of colon length (between about 25% and 35% increase), as compared to untreated mice (saline). Furthermore, a combination of HA and sodium butyrate showed a statistically significant colon length enhancement, compared to untreated mice.

Furthermore, at day 11 after treatment the inventors observed a remarkable improvement in the DAI score of mice treated with CBD and HA (group 3), as compared to control treatments with saline and HA (groups 1 and 2).

To this end, a combined treatment with CBD and HA or alternatively with CBD and SCF (and optionally HA) showed a significant treatment efficient in-vivo. Accordingly, it is presumed that these compositions/combinations can be successfully implemented for treated human subject afflicted with a disease or disorder associated with rectal and/or intestinal inflammation, as disclosed herein.

While the present invention has been particularly described, persons skilled in the art will appreciate that many variations and modifications can be made. Therefore, the invention is not to be construed as restricted to the particularly described embodiments, and the scope and concept of the invention will be more readily understood by reference to the claims, which follow.

1. A composition comprising (i) a cannabinoid, and (ii) a compound comprising a short chain fatty acid (SCF), a salt thereof or both; wherein:
wherein a w/w ratio of said SCF to said cannabinoid within said composition is from 1000:1 to 1:1.

2. The composition of claim 1, wherein said cannabinoid comprises at least one of: cannabidiol-C4 (CBD-C4), cannabidiol (CBD-C5), cannabidiol momomethyl ether, cannabidiolic acid (CBD-A), cannabigerolic acid (CBN-A), cannabigerol (CBG), cannabinol (CBN), cannabinolic acid (CBN-A), cannabichromenic acid (CBC-A), cannabichromene (CBC), cannabidivarin, cannabidiorcol, and cannabidivarinic acid or any combination thereof.

3. The composition of claim 1, wherein said SCF comprises butyric acid, a propionic acid or both including any salt thereof.

4. The composition of claim 1, wherein at least 90% w/w of the total cannabinoid content is CBD-C5.

5. The composition of claim 1, wherein a weight per weight (w/w) concentration of said cannabinoid within said composition is from 0.1 to 50%.

6. The composition of claim 1, wherein a w/w concentration of said SCF within said composition is from 0.1 to 99%, and wherein a w/w ratio of said SCF to said cannabinoid within said composition is from 100:1 to 3:1.

7. The composition of claim 1, further comprising an additional compound comprising an HA component, a fructan including any salt or any combination thereof.

8. The composition of claim 7, wherein said HA component comprises at least one of: HA, a derivative of HA, a salt of HA or any combination thereof.

9. The composition of claim 8, wherein said HA has an average molecular weight ranging from 1000 to 10⁶ Da.

10. The composition of claim 7, wherein said fructan comprises inulin, a derivative thereof or both.

11. The composition of claim 7, wherein a molar ratio of (i) said additional compound to (ii) said cannabinoid within said composition is from 10.000:1 to 10:1.

12. (canceled)

13. The composition of claim 7, wherein a w/w ratio of (i) said cannabinoid to (ii) said HA component within said composition is from 0.1:20 to 1.1.

14. A pharmaceutical composition, comprising the composition of claim 1 and a pharmaceutically acceptable carrier.

15. (canceled)

16. (canceled)

17. (canceled)

18. (canceled)

19. A kit comprising a first composition comprising a cannabinoid and a second composition comprising a SCF, a salt thereof or both.

20. The kit of claim 19, wherein said SCF comprises butyric acid, a propionic acid or both including any salt thereof.

21. The kit of claim 19, wherein at least 90% w/w of a total cannabinoid content within the first composition is CBD-C5.

22. (canceled)

23. (canceled)

24. (canceled)

25. The kit of claim 19, wherein the first composition, the second composition or both further comprise an HA component, a fructan or both, including any salt thereof.

26. (canceled)

27. (canceled)

28. A method for preventing or treating a disease or a disorder associated with vaginal, rectal and/or intestinal inflammation in a subject in need thereof, comprising administering to the subject the pharmaceutical composition of claim 14.

29. The method of claim 28, wherein said administering is by a topical administration, a rectal administration or both.

30. The method of claim 28, wherein said disease or disorder is selected from pouchitis, inflammatory bowel disease, inflammation of the distal ileum, inflammation of the terminal ileum or any combination thereof.
